(19) United States
(12) Patent Application Publication
(10) Pub. No.: US 2005/0169912 A1
(43) Pub. Date: Aug. 4, 2005

(54) Immunogenic peptides and uses thereof

(76) Inventors: Emanuel Calenoff, Alameda, CA (US); Charles Ditlow, Chicago, IL (US)

Correspondence Address: BARNES & THORNBURG, P.O. BOX 2786, CHICAGO, IL, 60690-2786, US

(21) Appl. No.: 10/979,890

(22) Filed: Nov. 2, 2004

Related U.S. Application Data
(62) Division of application No. 09/838,967, filed on Apr. 20, 2001, now Pat. No. 6,670,537.
Application No. 09/838,967 is a continuation-in-part of application No. 09/837,630, filed on Apr. 18, 2001, now abandoned.

(51) Int. Cl. A61K 39/395; A61K 39/00; G01N 33/53; C07K 14/47; C07K 16/18

(52) U.S. Cl. 424/130.1; 530/350; 530/388.1; 424/185.1; 435/7.1

(57) ABSTRACT

Novel general methods and compositions provide specific or highly associated antigenic peptides useful for diagnosis and treatment of diseases caused by microorganisms, allergens or other proteins. Specific or highly associated peptide antigens are also useful for diagnosis, treatment and prevention of auto-immune diseases, allergens and other diseases.

This application claims priority from co-pending U.S. Ser. No. 09/837,630 filed on Apr. 18, 2001.

### BACKGROUND

This invention relates to novel general methods and compositions that provide functionally specific immunogenic peptides (peptide antigens) useful for diagnosis and treatment of autoimmune diseases, allergies and diseases caused by microorganisms.

Immunological methods (assays) have been reported to detect the presence of specific disease-causing or allergy causing organisms in biological fluids. Diagnostic kits comprising antigen, antibody, label and anti-antibodies have been described based on some of the assays. Antigens used as indicators of agents causative of, or associated with diseases or conditions have included peptides derived from proteins in the agents. Heliobacter pylori, an organism associated with gastrointestinal disease including gastric cancer, is one specific target of such assays.

Therapies and vaccines have been proposed including use of peptides to immunize. Casadevall and Scharff (1994) proposed antibody-based therapies to overcome some of the problems and limitations of antibiotic therapy. Shigeoka et al. (1984) reported protective efficacy of hybridoma type-specific monoclonal antibody against streptococcus in an experimental model (passive immunotherapy). Other potential targets are autoimmune diseases such as rheumatoid arthritis for which oral desensitization by heat shock protein 60 from E. coli has been reported. Ben-Yedidia et al. (1999) reported that whole virus or surface glycoproteins “fail to induce broad specificity protection” from influenza infection. They developed a human peptide-based recombinant vaccine based on epitopes “recognized by prevalent HLAs.” It was tested in a human/mouse radiation chimera. The authors concluded “further investigation is needed to establish the efficacy of such a peptide vaccine in human subjects.” Adjuvant was not needed.

Complete, long-term and cross-strain immunity is needed for vaccines. Conserved regions of peptides across strains may be desirable for this purpose. Javed et al (1995) reported that small differences in the amino acid sequences of peptides, even single amino acid differences, for T cell-mediated autoimmune disease, e.g. multiple sclerosis, may have different protective effects when administered to confer oral tolerance. Although related to specific diseases or conditions peptides have been reported, no systematic way is available to identify candidates for diagnostic and therapeutic peptides. Algorithms designed to select optimal peptides could enhance specificity of the immunoassays and therapies employing the peptides.

### SUMMARY OF THE INVENTION

The invention relates to general methods and compositions that provide immunogenic peptides useful for diagnosis and treatment of diseases and conditions wherein a causative organism, agent or tissue has at least one identifiable protein component. Such targeted diseases include autoimmune diseases, diseases caused by microorganisms, and allergic conditions. The protein is designated a “target” protein. In some embodiments, target proteins are associated with a disease or condition, even though causation is not established.

An aspect of the invention is algorithms for determining, selecting and/or constructing synthetic peptides that are candidates for producing an immune response useful in the diagnosis and treatment of such diseases, e.g. autoimmune diseases, diseases caused by microorganisms and allergic conditions. Suitable peptides of the present invention are functionally specific for target proteins causative of, or associated with, a targeted disease or condition. That is, when detected immunologically the peptides are indicators of the target proteins, and consequently, of the disease or condition caused by, or associated with, the target protein. Although by definition, peptides of the present invention are functionally specific, they are not structurally specific, because the peptides match not only amino acid sequences of target proteins, but also to some degree match sequences of non-target proteins. The non-target proteins are used for amino acid sequence comparison to identify peptides suitable for practice of the present invention. However, by means of the peptide selection algorithms of the present invention, homology to non-target proteins does not interfere with functional specificity, in particular, when a plurality of peptides are used to identify targeted organisms or tissues, or are used to formulate treatments. Peptides of the present invention may be derived from parent (target) proteins by e.g. enzymatic digestion or be made synthetically by methods known to those of skill in the art, e.g. automated solid phase methods.

The invention is generally directed to immunogenic peptides which include (a) a sequence of at least 4 to about 100 amino acids; (b) a net hydrophilic structure as determined by the amino acid sequence of the peptide of the target protein associated with, or causative of, the disease or condition of interest; (c) a net amino acid sequence homology of less than 50 percent as compared to the structure of peptide regions on proteins of related non-target proteins (the “comparative” proteins); (d) an amino acid sequence wherein no more than three amino acids that are identical in structure and position to amino acid sequences of the comparative proteins, adjoin one another; and (e) an antigenic profile which elicits a highly specific, antibody-reactive or an immune cell reactive immune response. Arrays of peptides used to detect or treat a targeted organism or tissue include discriminating pluralities of peptides selected by methods of the present invention.

Peptides selected by the methods of the present invention are preferably small, e.g. from 4 to about 100 amino acids in length. More preferred lengths of the peptides are from 4-7 amino acids or 4-10, or 5-10, amino acids, although peptides up to about 25 or to 100 amino acids in length, are also within the scope of the invention. The peptides have a net hydrophilic structure located on the surface of a target molecule (protein) from which they are derived or are synthesized from knowledge of the target molecule. The peptides or fragments thereof include any variation in the amino acid sequence, whether by conservative amino acid substitution, deletion, or other processes, provided that the polypeptides are in accord with the functional criteria of the present invention. More specifically, more than one peptide, the sequences of which are in accord with the criteria of the present invention, are preferably present to enhance the discriminatory power of the immunoassays and therapies disclosed herein. That is, a plurality of peptide antigens forms an array (or repertoire) of molecules suitable for diagnosis and treatment of autoimmune diseases, allergenic diseases and diseases caused by microorganisms.

Non-target proteins are selected for comparative purposes, by scanning for all available sequence matches in computer data banks. Amino acid sequences of at least 4 in length are selected from at least 1 of the protein sequences that showed some degree of homology to the target protein. Closest matches are preferred.

The steps outlined in Table 1 lead to the production of peptides suitable for the practice of the present invention.

TABLE 1
Steps in Obtaining Immuno-Specific Peptides

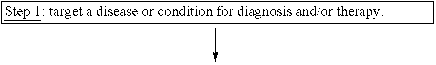

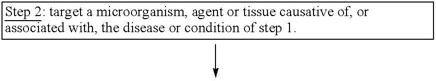

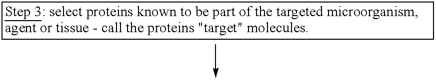

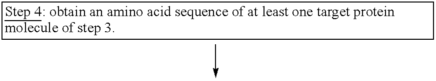

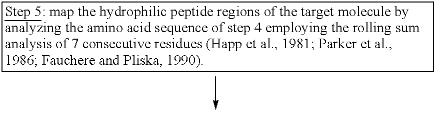

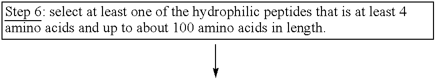

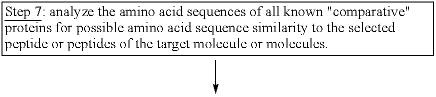

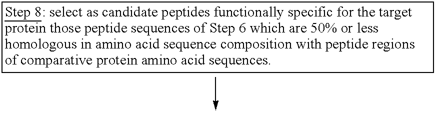

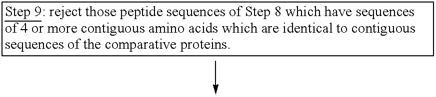

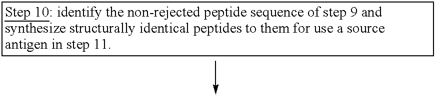

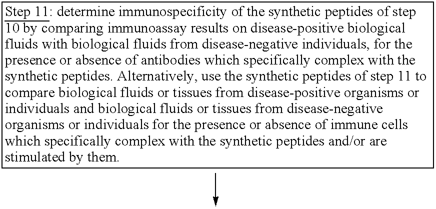

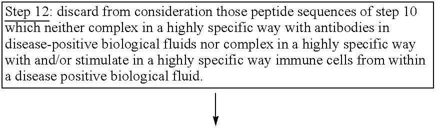

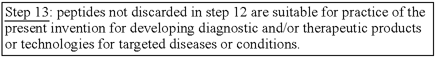

To reiterate, for the methods of the present invention, a disease or condition is targeted, for which an organism, agent or tissue is identified that is known to be causative of, or associated with, the targeted disease or condition for which diagnosis and/or treatment is sought. Proteins from the organism, agent or tissue are selected from databases, e.g. the NIH gene bank, which is available on the internet. These proteins are called “target” proteins. Functionally specific peptide antigen candidates are identified from within the amino acid structure of each protein on the basis of being hydrophilic and therefore likely to be on the outer surface of the protein. The amino acid structure of the candidate peptides are then compared to the amino acid structures found in individual non-target, (non-specific), proteins by using computer matching programs such as BLAST. Functionally specific peptide antigens are selected on the basis of having no more than 50% amino acid matching (sequence homology) with the comparative protein peptide sequences. Furthermore, whatever candidate antigen sequences satisfy this criteria must also possess no more than three contiguous (immediately adjacent to one another) amino acids which are sequentially homologous to amino acids matching foreign protein amino acid sequences.

The present invention includes peptides which function as specific peptides for target proteins. Suitable peptides include the following structure:
(a) four to one hundred amino acids in length;
(b) a net hydrophilic structure as determined by their amino acid sequences;
(c) a net sequence homology of 50 percent or less as compared to the structure of single non-specific proteins, that is proteins from non-target microorganisms, or proteins from non-target tissues;
(d) an amino acid sequence wherein no more than three contiguous amino acids are homologous to contiguous amino acids on a non-target protein adjoin one another; and
(e) an antigenic profile which elicits a highly specific, antibody-reactive immune response and/or highly specific, cellular immune response.

A method for identifying candidate microorganism-specific or microorganism-associated peptide antigens, or peptide from allergens or tissue specific immunogenic peptides, includes the steps of:
(a) obtaining an amino acid sequence of a protein representative of the microorganism, allergen or tissue;
(b) mapping hydrophilic regions of the protein by analyzing the amino acid sequence of the protein employing the rolling sum analysis of 7 consecutive residues;
(c) selecting fragments of from 4 to 100 amino acids in length;
(d) fragments for (c) have a net sequence homology of 50 percent or less as compared to the structure of peptides of single non-target proteins, that is proteins comprising non-targeted microorganisms, or proteins comprising non-targeted tissues;
(e) an amino acid sequence wherein no more than three contiguous amino acids are homologous to contiguous amino acids on a non-target protein adjoin one another;
(f) synthesizing candidate peptides that fit the criteria of steps (a) to (e);
(g) labeling the peptides at either the NH₂ or COOH end of their amino acid sequence; and
(h) testing by means of immunoassays or immune cell proliferation assays whether the candidate peptides are specific or highly associated with specific infecting microorganisms or specific disease states.

An aspect of the invention is immunoassays employing the immunogenic peptides to measure specific peptide-reactive antibodies in biological fluids; more specifically, an aspect of the invention is monoclonal antibodies and antibody-like molecules such as Fab2 and FAb fragments, known to those skilled in the art, and recombinant and synthetic proteins thereof, which are specifically reactive with the immunogenic peptides of the present invention. Immunoassays employing these antibodies or antibody-like molecules of the present invention are used to measure in biological fluids, molecules containing peptide regions which correspond in vivo to the immunogenic peptides of the present invention.

An immunogenic composition capable of inducing a mammal to produce antibodies specific for an epitope on a protein representative of a microorganism or diseased tissue includes a peptide of the present invention. Synthetic recombinant vaccines that combine epitopes (e.g. different peptides) are contemplated. A method of producing immunity includes obtaining and administering an effective amount of the constructs including the peptide to a mammal, wherein “effective amount” is determined by methods known to those of skill in the art.

A molecule which is specifically reactive with a peptide of the present invention includes monoclonal antibodies or immunogenic fragments thereof, antibody-like recombinant proteins and antibody-like synthetic proteins or peptides.

An aspect of the present invention is a diagnostic method wherein a plurality of peptides of the present invention are placed in a microchip that are used to detect a target protein in a subject from which a biological sample is obtained. The target protein is detected by hybridization of antibodies in the biologic sample to the plurality of target peptides on the microchip.

A method of delivering microorganism molecules containing epitopes expressed by the peptides of the present invention for the purpose of identifying infectious status of a mammal, uses an immunoassay for the complexing of microorganism molecules with a molecule or immune cell that is specifically reactive with a peptide of the present invention.

Imaging reagents are also developed using labeled molecules of the present invention including antibodies or antibody-like molecules, directed toward peptides of the present invention. Suitable labels include radioisotopes, a paramagnetic label, and a water density label. The labels complexed with the antibodies or antibody-like molecules target specific microorganisms or specific tissues and respond to image detectors to identify their location.

The label may be radioisotopic which, upon binding to microorganisms or tissue highlights the presence of the microorganisms or tissues when scanned with a nuclear medicine scanner.

The label may be a paramagnetic label which, upon binding to molecules representative of microorganisms or tissues highlights the presence of the microorganisms or tissues when scanned with a nuclear magnetic resonance (NMR) scanner.

The label may be a water density label which, upon binding to molecules representative of the microorganisms or tissue highlights the presence of the microorganisms or tissues when scanned with a CAT scanner.

A therapeutic or preventive vaccine containing one or more immunogenic peptides of the present invention, and prepared by methods known to those skilled in the art of vaccine development, is an aspect of the invention. Generally, adjuvent/peptide conjugates including the immunogenic peptides coupled to molecules which facilitate enhanced immunogenicity, are used to stimulate the host immune system to facilitate the killing of microorganisms and thereafter maintain immune surveillance in case of reinfection. Alternatively, desensitization reagents can be prepared to treat autoimmune diseases and allergic diseases.

Vaccines created by recombinant techniques containing immunogenic peptides together with adjuvant molecular sequences which promote increased immunogenicity of the immunogenic peptides to stimulate the host immune system to facilitate the killing of microorganisms and thereafter maintain immune surveillance in case of re-infection, are also within the scope of the invention.

An aspect of the present invention are therapeutic agents comprising peptides of the present invention which are coupled to agents which kill immune cells in a host responsible for a disease process such as allergy or an autoimmune disease.

A therapeutic construct that includes a peptide of the present invention includes:
(a) adjuvant/peptide conjugates made of the peptide coupled to a molecule which facilitates enhanced immunogenicity; and
(b) neomolecules created by recombinant techniques containing a peptide with adjuvant molecular sequences which promote increased immunogenicity of the peptide.

The therapeutic construct may be a nucleic acid molecule having a nucleotide sequence that encodes a peptide of the present invention. The nucleic acid molecule is administered to the cells of an individual and then expressed by the individual's cells as a protein or peptide for the purpose of auto-stimulation of the individual's immune system.

A therapeutic construct for desensitizing a host suffering from an autoimmune disease or an allergy includes a peptide of the present invention wherein:
(a) the construct is initially administered in a dose insufficient to sustain or augment the autoimmune or allergic immune response; but
(b) as the construct dosage is steadily increased, the autoimmune or allergic immune process is abrogated or ameliorated.

Routes of administration of peptides include nasopharyngeal or respiratory delivery of soluble peptides, or subcutaneous injection.

### DEFINITIONS

“Functionally specific” refers to peptides that produce antibodies in biological fluids from patients known to have the targeted disease or condition, or complex in a specific way with and/or stimulate in a specific way, immune cells from a disease positive biological fluid, compared to reactions with disease negative biological fluid.

“Targeted” refers to diseases or conditions for which diagnosis or therapy is sought; “Target” refers to proteins under investigation as candidates for diagnostic or therapeutic use for the targeted diseases or conditions.

“Contiguous” amino acids are adjacent to each other in an amino acid sequence if more than 2 amino acids, there are no gaps between them in the sequence.

The term “antigen presenting cell” (APC) includes “professional antigen presenting cells” that constitutively express MHC class II molecules (e.g., B lymphocytes, monocytes, dendritic cells, Langerhans cells, and activated T cells in humans) as well as other antigen presenting cells that are capable of presenting antigen to T cells. APCs can express the appropriate combination of MHC molecules and costimulatory and/or adhesion molecules known in the art to be sufficient for presentation of antigen to T cells or can be induced or engineered to express such molecules.

As used herein, the term “immune response” includes T cell mediated and/or B cell mediated immune responses that are influenced by modulation of T cell costimulation. Exemplary immune responses include T cell responses, e.g., proliferation, cytoline production, and cellular cytotoxicity. In addition, the term “immune response” includes immune responses that are indirectly effected by T cell activation, e.g., antibody production (humoral responses) and activation of cytokine responsive cells, e.g., macrophages.

The term “markers,” as used herein, includes any molecule which is detectable in a biological sample and indicates the presence of another molecule of interest. Some markers are antigenic. Markers are useful because their presence is associated with a disease or condition of interest.

The single letter code for amino acids, well known to those of skill in the art, is used herein (see Table 2).

TABLE 2
Abbreviations for amino acids

| | Three-letter | One-letter |
|---|---|---|
| Amino acid | abbreviation | symbol |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamic acid | Glu | E |
| Glutamine or glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| unknown or other | Xaa | X |

### BRIEF DESCRIPTION OF THE DRAWINGS

FIG. 1 illustrates the amino acid sequence of the flagellar sheath adhesion protein of Helicobacter pylori; the bolded and underlined portions of the sequence are portions that are hydrophilic and likely to be expressed on the surface of the folded protein.

FIG. 2a shows one of the hydrophilic peptide regions of the flagellar sheath adhesion protein of Helicobacter pylori in alignment with closely matched peptide sequences of two comparative microoganismal proteins, the Streptococcus pneumoniae, pspA protein and the Mycoplasma hominis Lp1 protein; the pspA and Lp1 proteins were those most closely matching the linear amino acid sequence of the Helicobacter pylori flagellar sheath adhesion protein sequence using the BLAST amino acid sequence homology comparison program on the National Library of Medicine web site [www.ncbi.nlm.nih.gov:80/BLAST/-]; only amino acids that are identical to H. pylori protein sequence, are shown.

FIG. 2b shows three boxes drawn around different constituent sub-sequences of the Helicobacter pylori flagellar adhesion sheath peptide sequence; the Helicobacter pylori amino acid sequence with the bold lined box is likely to serve as a functionally specific antigen when compared to the two aligned, comparative protein amino acid sequences using the selection criteria of the disclosed invention; results using a peptide with this sequence shown in FIG. 3; the Helicobacter pylori sequence within the second, lightly lined box also satisfies the selection criteria of the present invention; results using a peptide with this sequences are shown in FIG. 4; whereas the Helicobacter pylori sequence within the third, dashed line box does not satisfy the selection criteria and would be discarded or rejected as a candidate; results using this are shown in FIG. 5.

FIG. 3 is a graphical representation of results of using the Helecobacter pylori peptide sequences MQEIDKKLTQKN shown in FIG. 2b as a source antigen peptides used to complex with antibody in sera from 30 Helicobacter pylori infected patients and from 30 healthy control subjects; the dotted line at the bottom of the plotted results represents the positive/negative threshold of the immunoassay using the control mean plus 2/5 standard deviations; this peptide identified three Helicobacter pylori infected individuals from within a group of thirty; no control sera were incorrectly identified as positive for the peptide as determined by antibodies to the peptide.

FIG. 4 is a graphical representation of results of using the Helicobacter pylori peptide sequence QKDAKECKGKRN shown in FIG. 2b as a source peptide antigens used to complex with antibody in sera from 30 Helicobacter pylori infected patients and from 30 healthy control subjects; the dotted like at the top of the plotted results represents the positive/negative threshold of the immunoassay using the control mean plus 2.5 standard deviations; this peptide does not serve to identify Helicobacter pylori infected individuals from within a group of thirty in spite of satisfying most of the selection criteria of the described invention, thus confirming the need to test specific functional utility (immunogenic) of the peptide antigen.

FIG. 5 is a graphical representation of results of using the Heicobacter pylori peptide sequence QKDAKELKGKRN shown in FIG. 2b as a source antigen used to complex with antibody in sera from 30 Helicobacter pylori infected patients and from 30 healthy control subjects; the dotted line at the center of the plotted results represents the positive/negative threshold of the immunoassay using the control mean plus 2.5 standard deviations; as expected, this peptide does not serve to identify Helicobacter pylori infected individuals from within a group of thirty because its structure fails the basic selection criteria of the present invention.

FIG. 6 lists additional functionally specific Helicobacter pylori antigens which satisfy all of the criteria of the present invention; these peptides were derived from different H. pylori targeted proteins shown in FIGS. 2a and 2b.

FIG. 7 summarizes the diagnostic capability made possible by testing (a) patient; and (b) control sera against a plurality of 14 individual, specific peptides using immunoassays incorporating the peptides listed in FIG. 6.

FIG. 8 lists functionally specific collagen type II antigens which satisfy all of the listed criteria of the described invention; type II collagen is one of several collagen types known to be associated with rheumatoid arthritis (He, 2000; Morgan,1987).

### DESCRIPTION OF THE INVENTION

The invention relates to methods and compositions for obtaining specific or highly associated antigens (generally antigenic peptides) for use in diagnosis and treatment of autoimmune diseases and diseases caused by, or associated with microorganisms, allergens, or tissues. Target proteins are selected from the microorganism, allergen or tissue, and are used to further select immunogenic peptides. An aspect of the invention is algorithms for determining, selecting and/or constructing peptide antigens (immunogenic) that are suitable for use in diagnostic tests, in producing specific antibodies for use in diagnosis or treatment, and producing immunogenic constructs for treatment of the targeted diseases or conditions. Aspects of the invention include a large repertoire (array) of specific and associated peptide antigens.

A disease or condition for which a microorganism, agent or tissue having a protein component, is causative of, or associated with, is targeted for diagnosis and therapy. At least one protein known to be in the microorganism, agent or tissue is identified and targeted.

Computer-aided searches are performed to locate at least two amino acid sequences that show no more than 50% homology with the target protein. These sequences are designated non-targeted, non-specific or comparative proteins. For example, as shown in FIG. 2, a selected amino acid sequence representative of targeted microorganism Heliobacter pylori was a flagellar sheath adhesion protein sequence. Amino acid sequences for proteins from two other targeted microorganisms are shown that were selected by using the BLAST computer program to compare the H. pylori protein sequence with the NIH gene database. The pspA homologous sequence from Streptococcus pneumoniae and the Lpp1 homologous sequence from Mycoplasma hominis, sequences were compared with that of the targeted protein from H. pylori.

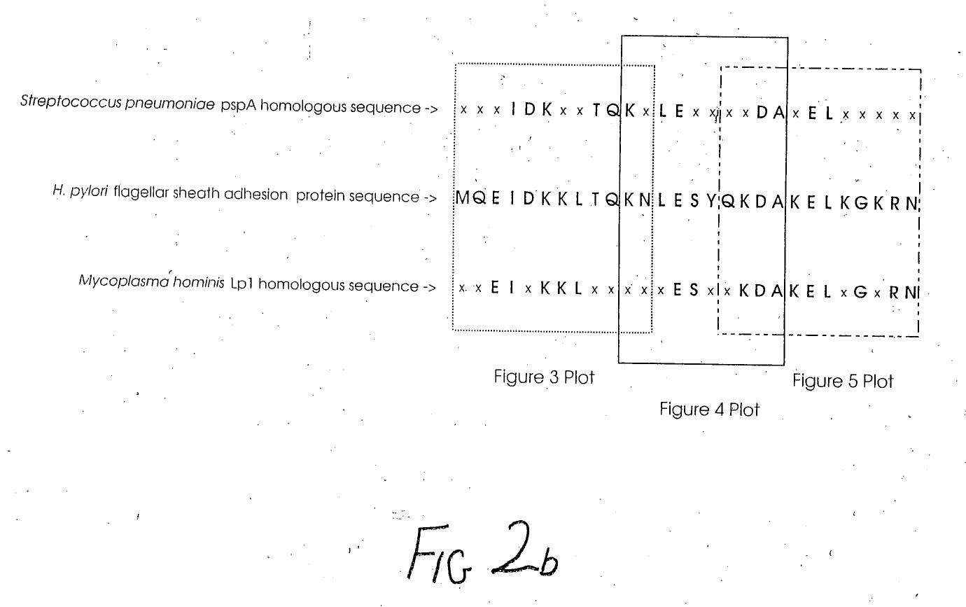

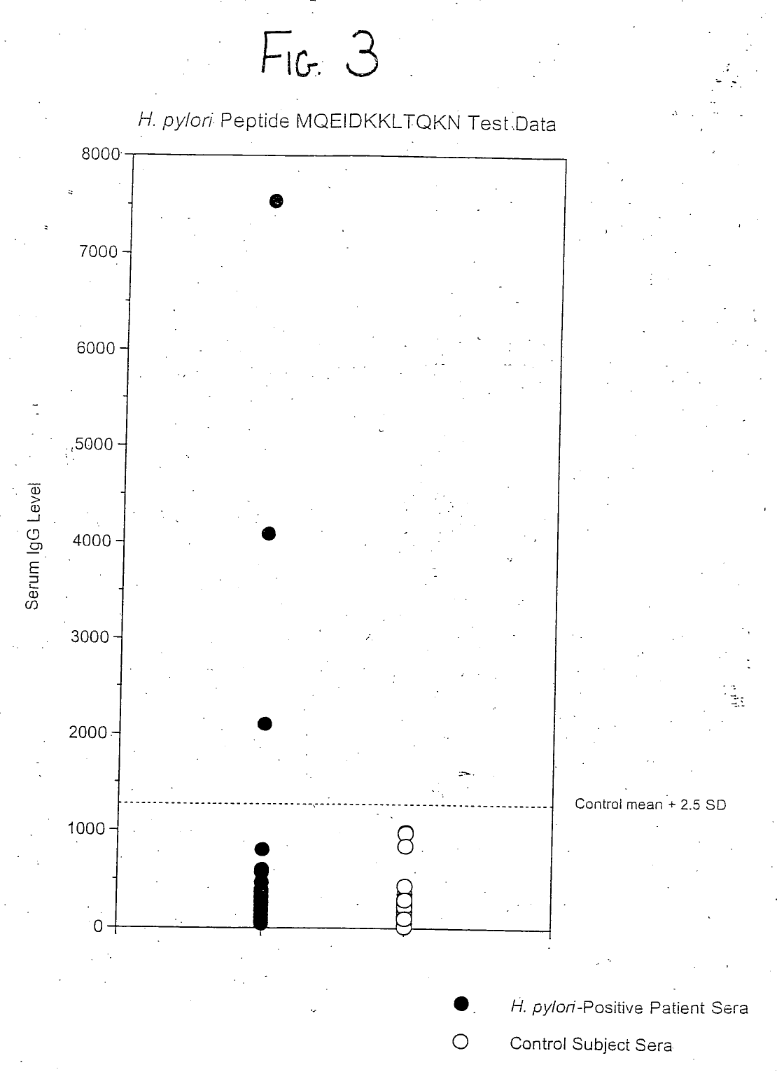

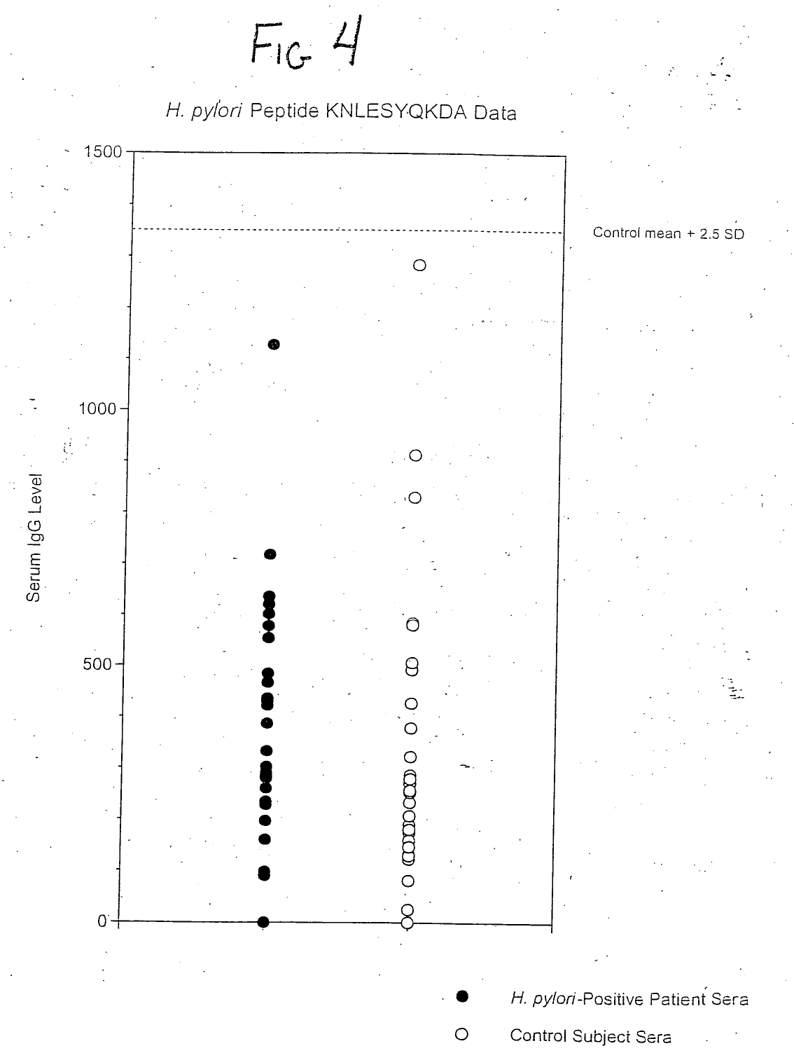

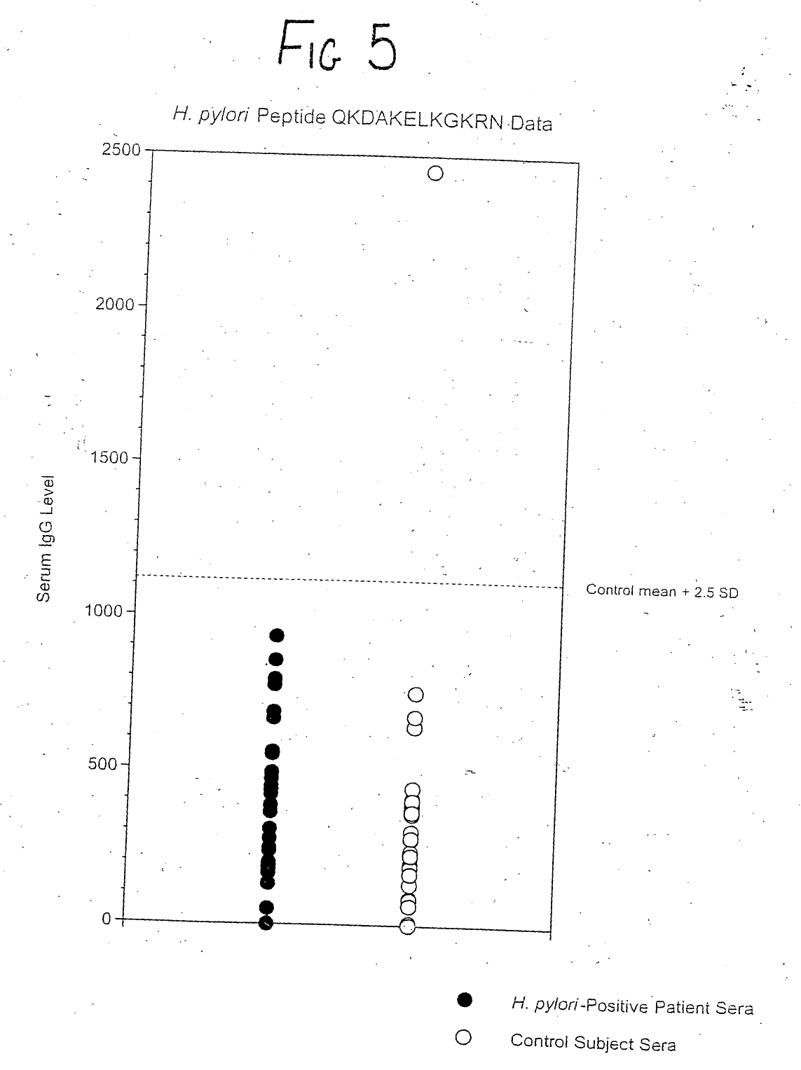

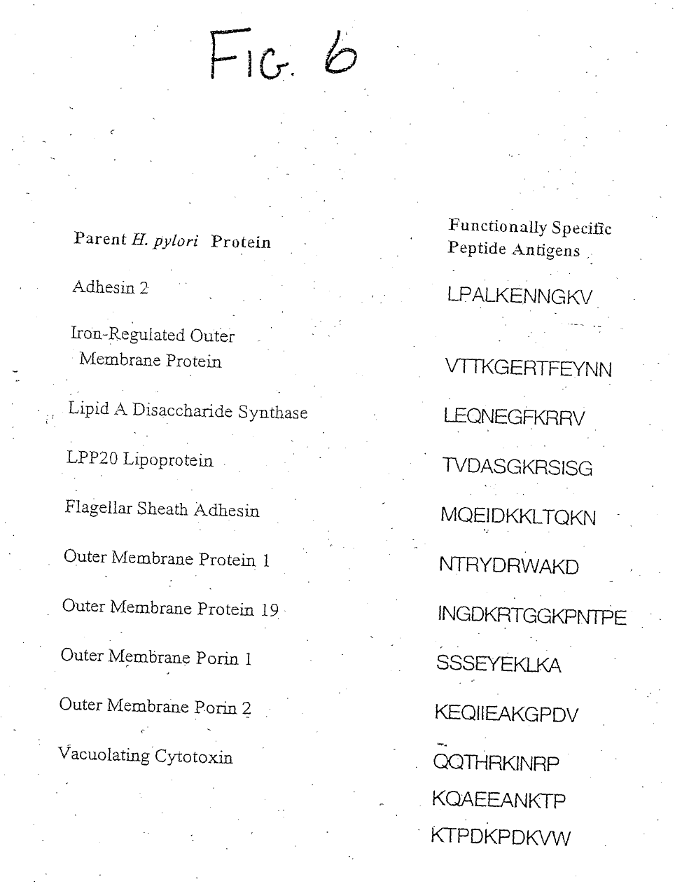

Comparison of the aligned amino acid sequences were made for the target, and at least 2 of the comparative proteins. A sequence of at least 4 amino acids became a sequence for a candidate peptide that could be specific for H. pylori. Candidate peptides have sequences that may be capable of immunologically distinguishing biological samples from diseased vs. non-diseased persons. For example, sequence MQEIDKKLTQKN is a candidate sequence that was tested; results are shown in FIG. 3. Sequence KNLESYQKDA is a candidate sequence that was tested, results are shown in FIG. 4. Sequence QKDAKELKGKRN is a candidate sequence that was tested, results are shown in FIG. 5. As can be seen from the test results, the candidate sequences in FIGS. 4 and 5 were not functionally specific for the H. pylori protein.

Using the same procedure that led to FIG. 3 sequences being found suitable for practicing the invention, other functionally specific peptides are shown in FIG. 6.

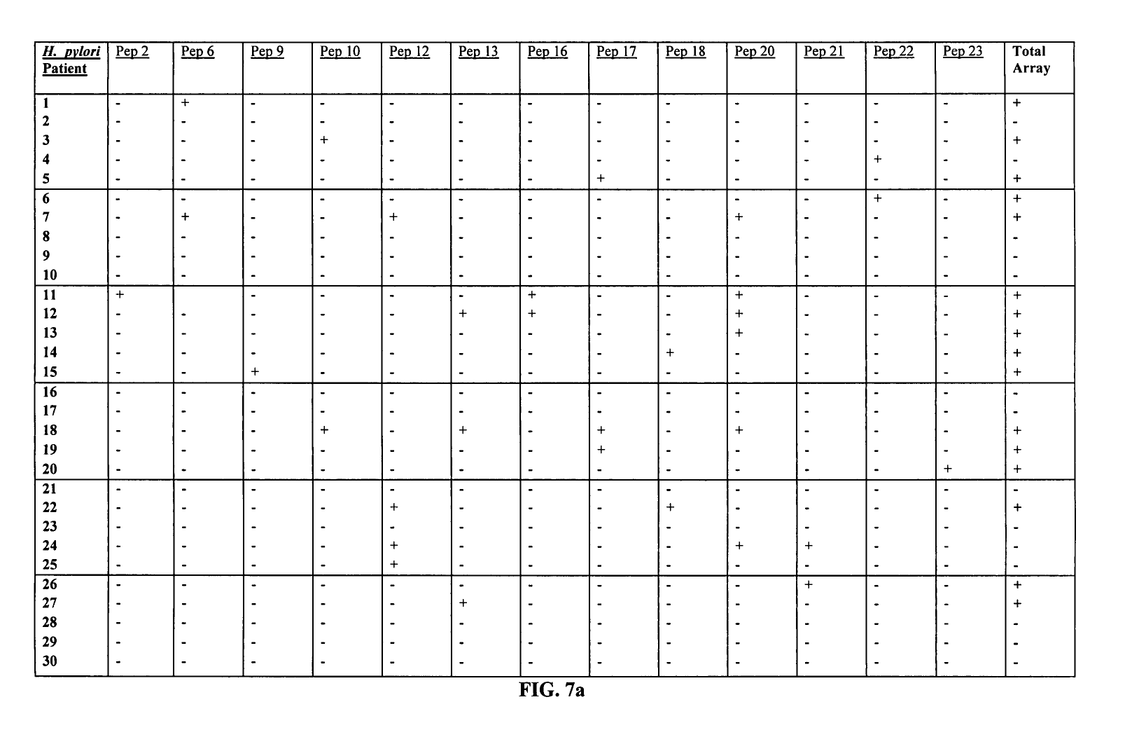

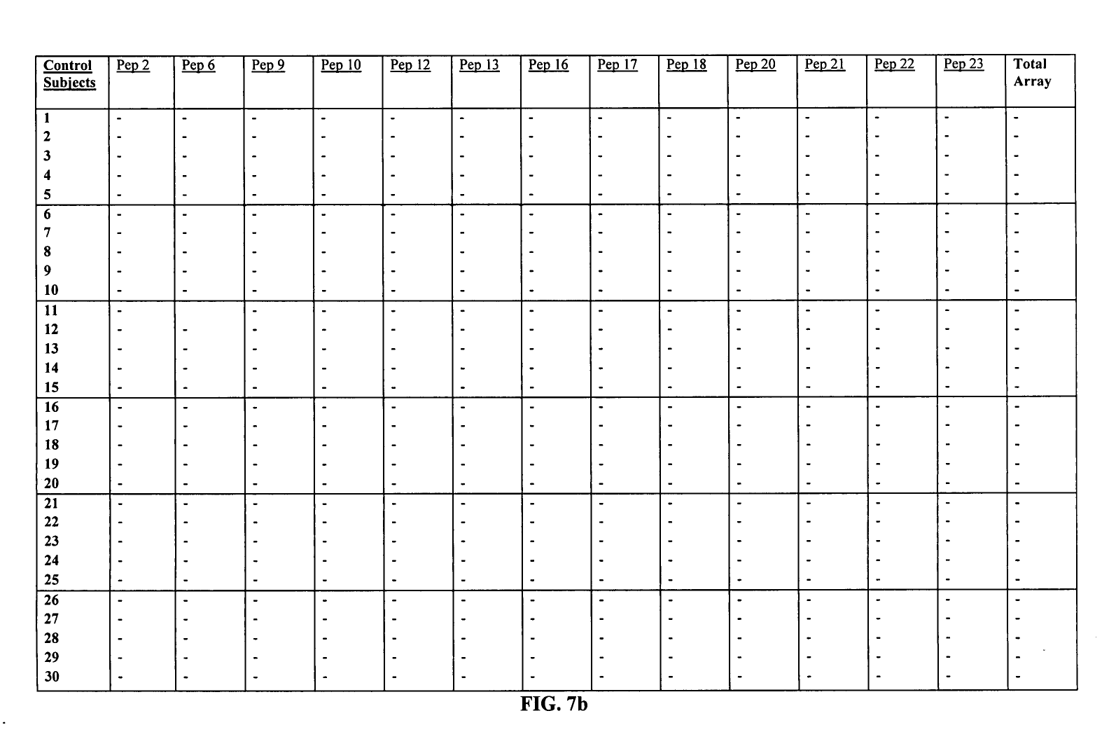

Preferably a plurality of peptides that satisfy the criteria for peptides specific for a targeted disease or condition, are used to develop a diagnostic test for individuals having a targeted disease or condition. This is illustrated in FIG. 7. In Table 3a, 16/30 persons known by other tests to be infected with H. pylori, tested positive for at least one individual peptide using IgG assays, whereas none (0) of 30 control subjects tested positive for any individual peptide using IgG assays.

Examples of therapeutic methods which can be formulated using a suitable antigen/marker array are (see Materials and Methods for details and citations) include:
a. passive immunization using constructs such as engineered antigen presenting cells and production of antigen presenting dendritic cells able to stimulate the host immune system to recognize and kill microorganisms or destroy or attenuate other foreign elements (allergens or tissues);
b. active immunization using vaccines including recombinant fusion proteins, vaccine compositions containing adjuvants, vaccine compositions containing nucleic acid molecules, recombinant microorganisms which express antigens of the present invention, antigen/antibody conjugates wherein the antibody acts as a delivery vehicle for targeting the antigen onto antigen presenting cells, and heat shock protein/antigen complexes;
c. cell lytic therapeutic antibodies, cell adhesion blocking antibodies, and growth factor receptor blocking antibodies.

Therapeutic methods using the peptide antigens of the present invention, either their amino acid sequences or the corresponding nucleic acid sequences that encode the peptides, include the following:
a. passive immunization using constructs such as engineered antigen presenting cells and production of antigen presenting dendritic cells able to stimulate the host immune system to recognize and kill microorganisms or destroy or attenuate other foreign elements;
b. active immunization using vaccines including recombinant fusion proteins, vaccine compositions containing adjuvants, vaccine compositions containing nucleic acid molecules, recombinant microorganisms which express suitable antigens, antigen/antibody conjugates wherein the antibody acts as a delivery vehicle for targeting the antigen onto antigen presenting cells, and heat shock protein/antigen complexes.

### EXAMPLES

The following examples are illustrative of the present invention.

### Example 1

### Detection of Helicobacter pylori in Biological Fluids of Persons Known to be Infected Compared to Biological Fluids from Non-Infected Persons

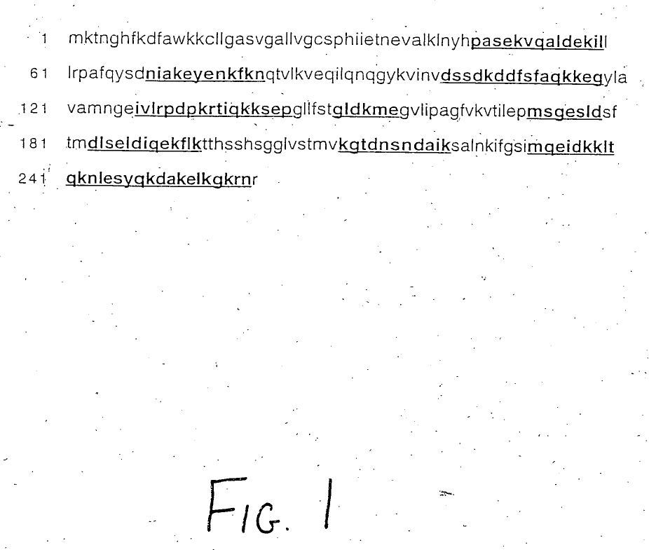

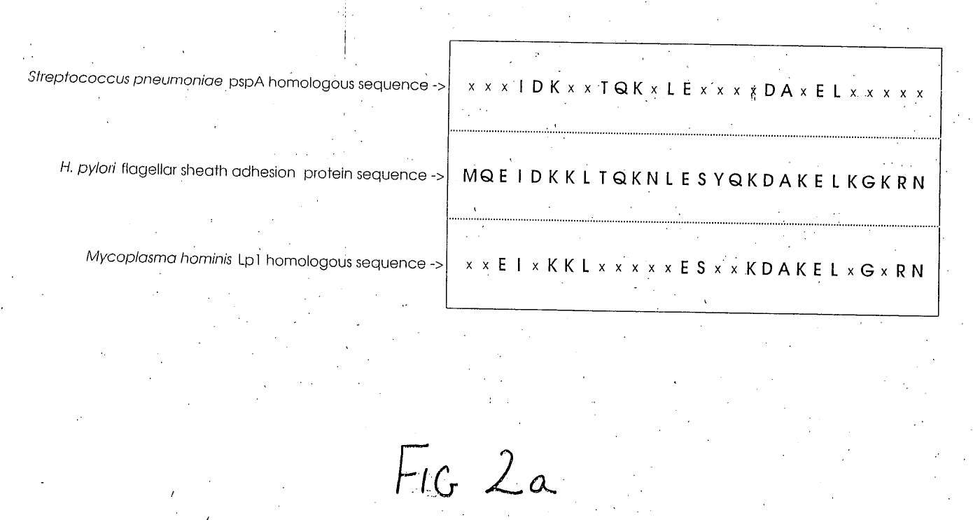

There are many proteins in the computer databases for H. pylori. A set of these proteins are shown in FIG. 6. Peptides selected from one of these proteins, the flagellar adhesion sheath proteins, (FIG. 1) are shown in FIGS. 2a and 2b. Synthetic peptides may be made by an automated synthesizer. Results using these peptides are shown in FIGS. 3, 4 and 5. As can be seen, the peptides of FIGS. 3 or 4 both fit the criteria of the present invention, whereas only the peptide of FIG. 3 detected fluids from infected persons. Therefore, candidate peptides selected by algorithms of the present invention, must be confirmed as specific immunogens.

### Example 2

### Detection of Rheumatoid Arthritis by Use of Peptides from Type II Collagen

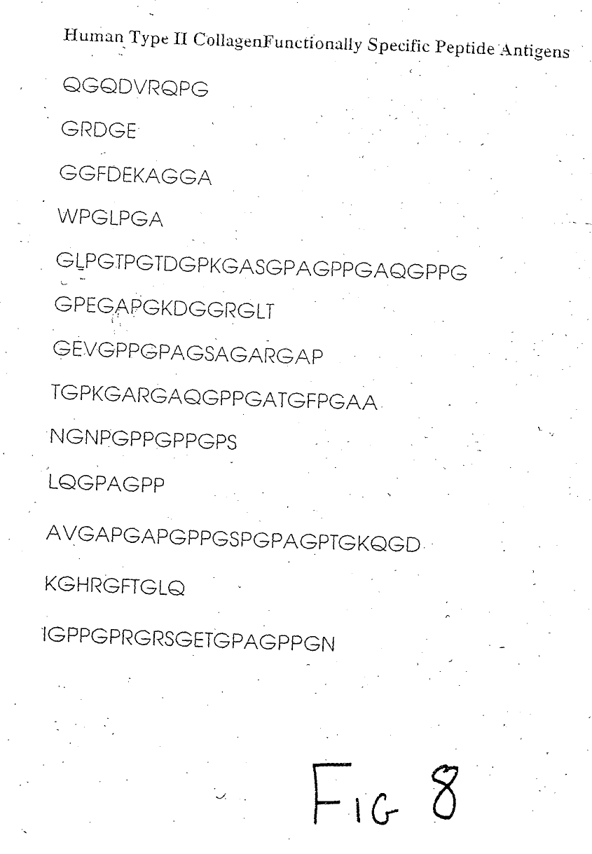

FIG. 8 shows peptides from Type II Collagen (Trentham et al., 1993) that can be used to detect biological fluids from person who are affected with rheumatoid arthritis by the methods and compositions of the present invention.

Materials and Methods

1. Possible Outcomes for Peptides Screened as Antigens in Serum Antibody Assays:

1. A positive result indicating the presence of a peptide-specific antibody in patient biological fluid samples, absent evidence of antibody in samples from subjects without the microorganism or autoimmune disease (FIG. 3) indicates the tested peptide is a specific peptide (immunogen) for a targeted protein.

2. A significantly higher positive prevalence of a peptide-specific antibody in patient biological fluid samples as compared to samples from subjects without the microorganisms or autoimmune disease (FIG. 4) indicates either that the tested peptide is specific and that the few control positives are asymptomatic or that the peptide serves as a highly associated antigen.

3. No difference in positive antibody levels between patients and subjects without disease. Peptides producing these results are neither specific nor highly associated with a microorganism or autoimmune disease (FIG. 5).

2. Immunoassay Method 1: Used to Detect Serum IgA, IgD, IgE, IgG, and IgM Antibodies Specific for Individual Peptide Antigens

Materials:

NeutrAvidin^{a} conjugated paper disc, 6 mm.

Serum diluent: 10 mM sodium phosphate, pH 7.20, with 150 mM sodium chloride, and 0.20 mg/mL sodium azide.

NeutrAvidin^{a} coated white microtiter plate, stored in 10 mM Tris-HCL, pH 7.50, containing 600 mM sodium chloride and 0.2 mg/mL thimerosal.

Plate blocking solution: 10 mM sodium phosphate, pH 7.20, containing 150 mM sodium chloride, 0.5 mg/mL Triton X-405 and 0.2 mg/mL thimerosal.

Plate wash buffer: 20 mM Tris chloride, pH 7.4, containing 150 mM sodium chloride, 0.5 mg/mL triton x-405 and 0.2 mg/mL thimerosal.

Peptide solution: 0.06 μg/mL peptide dissolved in 20 mM Tris chloride, pH 7.4, containing 600 mM sodium chloride, 30 mg/mL polyethylene glycol 4000, 1 mm ethylenediaminetetraacetic acid, 1 mM ethylene glycol-bis(§-aminoethyl ether)N,N,N′,N′-tetraacetic acid, 0.5 mg/mL triton x-405 and 0.2 mg/mL thimerosal.

Control peptide solution: 0.013 μg/mL control peptide dissolved in 20 mM Tris chloride, pH 7.4, containing 600 mM sodium chloride, 30 mg/mL polyethylene glycol 4000, 1 mm ethylenediaminetetraacetic acid, 1 mM ethylene glycol-bis(§-aminoethyl ether)N,N,N′,N′-tetraacetic acid, 0.5 mg/mL triton x-405 and 0.2 mg/mL thimerosal.

Conjugate solution: 0.100 μg/mL alkaline phosphatase conjugated polyclonal goat anti human IgG dissolved in 20 mM Tris-HCL, pH 7.40, with 600 mM sodium chloride, 30.0 mg/mL peg-4000, 3.0 mg/mL BSA, 0.5 mg/mL triton x-405 and 0.20 mg/mL thimerosal.

Substrate solution: 25.2 μg/mL 4-methylumbelliferyl phosphate dissolved in 180 mM 2-amino-2-methyl-1-propanol, pH 9.50, containing 123 μM magnesium chloride.

Serum preparation:
1. Add 100 μl serum to 15 neutravidin^{a} coated paper discs in a suitably sized test tube.
2. Incubate with gentle mixing at ambient temperature for 16-20 hours.
3. Add 7.9 mL of serum diluent and mix gently for 30 minutes.
4. Vortex the tube gently to completely release the serum from the discs.
5. Remove the treated serum from the discs and transfer it to a suitable storage tube.
6. Store the treated serum at 4 EC.

Assay procedure:
1. Two days before assay, aspirate the storage solution from the NeutrAvidin^{a} coated white microtiter plate and add 200 μl plate blocking solution to each well.
2. Cover the plate and incubate at ambient temperature for 16-20 hours.
3. One day before assay. Wash the blocked plate three times with plate wash buffer, approximately 275 μl per well per wash. Aspirate the final wash and add 100 μl peptide solution or 100 μl control peptide solution to the appropriate wells of the plate.
4. Cover the plate and incubate with gentle mixing at ambient temperature for 16-20 hours.
5. Day of assay, wash the blocked plate three times with plate wash buffer, approximately 275 μl per well per wash. Aspirate the final wash and add 100 μl treated serum to the appropriate wells of the plate.
6. Cover the plate and incubate at 25 EC for 2 hours.
7. Wash the blocked plate six times with plate wash buffer, approximately 275 μl per well per wash. Aspirate the final wash and add 100 μl conjugate solution to each assay well.
8. Cover the plate and incubate at 25 EC for 1.5 hours.
9. Wash the blocked plate six times with plate wash buffer, approximately 275 μl per well per wash. Aspirate the final wash and add 100 μl substrate solution to each assay well.
10. Read the plate at 30 and 60 minutes in a fluorescence microtiter plate reader set at 365 nM excitation and 450 nM emission.
   3. Biotinylation of Human Serum Albumin

Materials:

Human Serum Albumin: Sigma A 8763

Sulfosuccinimidyl 6-(biotinamido) Hexanoate: Pierce 21335

Tris base: Sigma T 1503

20 ml sodium phosphate, pH 7.2

100 mM sodium hydroxide solution

Procedure:

Human serum albumin is dissolved in phosphate buffer at a concentration of approximately 40 mg/mL. The protein concentration of the solution is determined by absorbance at 280 nM (1 mg/mL=OD280 of 0.58) or by the Lowry method.

Immediately prior to biotinylation, the pH of the albumin solution is adjusted to 8.5 by the addition of sodium hydroxide. Succinimidyl biotin is then added at a molar ratio of 50:1 (422 mg succinimidyl biotin per mg albumin). The reaction mixture is vortexed thoroughly and then mixed gently for 45 minutes at ambient temperature.

Reaction byproducts and unreacted biotin are removed by extensive dialysis against phosphate buffer. The biotinylated human serum albumin is stored at 4° C.

4. Preparation of Covalent Ready Cyanogen Bromide (CNBR) Activated Paper Discs

Materials:

paper discs: Schleicher and Schuell 53870

CNBr solution: 20 gm CNBr (sigma c6388)+600 mL distilled water

1M NaOH
0.05M NaHCO3
25%, 50%, 75%, and 100% acetone
Distilled Water
Dessicant packets.: Sigma S8394
Zip lock plastic bags

Procedure:

The following procedure is performed under a hooded, well ventilated environment. 20 gm paper discs are swelled in 200 mL distilled water at room temperature. Swelled paper discs are then added to 600 mL of CNBr solution while stirring. Bring up the pH of the stirring mixture to 10.5 and maintain at pH 10.5 until 100 mL of 1 m NaOH have been used up. Aspirate the resulting liquid and wash discs with 500 mL of NaHCO3 buffer for 2 minutes at room temperature. Repeat wash step ×12. Rinse discs twice with 500 mL distilled water. Rinse discs twice with 500 mL 25% acetone. Rinse discs twice with 500 mL 50% acetone. Rinse discs twice with 500 mL 75% acetone. Rinse discs twice with 500 mL 100% acetone. Aspirate last acetone wash solution and allow discs to dry under a running fume hood at room temperature. Store dried CNBr activated paper discs in zip lock plastic bags containing dessicant packettes.

5. Preparation of NeutrAvidin Conjugated Paper Discs and Biotinylated Human Serum Albumin Conjugated Paper Discs

Materials:

Biotinylated human serum albumin: Prepared by method of Example 1

NeutrAvidin: pierce 31000

CNBr-activated paper discs: Prepared by method of Example 2

Modified Coca's buffer: 0.05M NaHCO3+0.15M NaCl. PH 7.2

0.05M ethanolamine solution

0.21M sodium acetate buffer, pH 4.0.

Paper disc incubation buffer: 0.05M sodium phosphate+0.15M NaCl+0.05% NaN3+0.5% Tween20

Procedure:

A 2.5 mg/mL solution of neutravidin is prepared in modified Coca's buffer. A 2.5 mg/mL solution of biotinylated human serum albumin is prepared in modified Coca's buffer. 50 CNBr-activated discs are added to each mL of protein solution. Each protein/disc mixture is agitated for 16 to 18 hours at room temperature. Each solution surrounding the respective paper discs is aspirated and each set of discs are washed ×3 with modified Coca's buffer. The washed discs are immersed in 0.05M ethanolamine solution and agitated for 3 hours in order to block any unreacted CNBr sites. Each set of paper discs is then washed ×3 with the sodium acetate buffer. During the third step, the paper discs are incubated in the sodium acetate buffer for 30 minutes under gentle agitation. Each set of paper discs is then washed ×4 in Coca's buffer and then stored in the paper disc incubation solution at 4° C.

6. Preparation of Neutravidin Coated Microtiter Plates

Materials:

Amino Polystyrene Microtiter Plates (White): Nunc 453686 or the equivalent

NeutrAvidin: pierce 31000

Disuccinimidyl suberate (DSS): pierce 21555

Dimethyl sulfoxide (DMSO): Burdick and Jackson 081-1

20 mM sodium phosphate, pH 5.5

50 mM sodium carbonate, pH 9.6

PBS with sodium azide.

Procedure:

Prepare a volume of neutravidin appropriate for the number of plates to be coated. The coating solution contains 20 mg/mL neutravidin in 20 mM sodium phosphate, pH 5.50.

Prepare a suitable volume of dss, 1.22 mg/mL, in dry DMSO. This solution must be used within 2 hours of preparation.

For each plate to be coated, add 60 mL DSS solution to each well followed by 60 mL of 50 mM sodium carbonate, pH 9.6. Incubate this mixture in the wells for 36 minutes at ambient temperature. Aspirate the wells and wash twice with deionized water. Immediately add 100 mL neutravidin solution. Cover the plate and incubate at ambient temperature for 16-18 hours.

Aspirate the coating solution and add approximately 280 mL PBS with azide to each well. Seal the plate with a foil cover. Store the coated plates at about 4° C.

7. Vaccination Methods

Methods for preparing and administering a vaccine using peptides as immunogens have been reported for preventing microorganismal infection.

Methods for preparing and administering a vaccine using peptides as immunogens have been reported for treating microorganismal infection. For example, O'Brien-Simpson et al. (2000) used the RgpA-Kgp complex from Porphyromonas gingivalis with incomplete Freund's adjuvant (IFA) protected against challenge by the microorganisms in mice. Peptide vaccine candidates were synthesized from the complex. These peptides were synthesized using standard, solid-phase protocols for 9-fluroenylmethoxy carbonyl chemistry with S-acetylmercaptoacetic acid (SAMA) as the N-terminal residue. The SAMA-peptides were then conjugated to diphtheria toxoid and used with IFA to immunize ALB/c mice. Both active-site peptides and three of the five ABM peptides gave protection (P<0.005) against challenge with P. ginigivalis in a murine lesion model.

Escalating doses of a vaccine consisting of a 9-amino acid peptide from aminoc acids 12-20 encoded by the E7 gene of HPV emulsified with incomplete Freud's adjuvant and in some patients with another peptide with a lipid tail, show promising results but the authors suggested that “further refinements of an HPV vaccine strategy to boost antigen specific immunicty should be explored” (Muderspach et al., 2000).

8. Passive Immunization Constructs that the Lost Immune System Recognizes and Kills Cells

1. Engineered antigen presenting cells

2. Dendritic cells

See the “Detailed Description of the Invention” and “Examples 1-7” from U.S. Pat. No. 5,871,156 and the “Detailed Description of the Invention” and “Examples 1-5” from U.S. Pat. No. 6,080,409, incorporated by reference.

9. Active Immunization Using Vaccines

1. Ben-Yedidia T, Marcus H, Reisner Y, Arnon R. Intranasal administration of peptide vaccine protects human/mouse radiation-chimera from influenza infection. Int. Immunol. July 1999;11(7):1043-51.

2. Adjuvants

See U.S. Pat. Nos. 5,750,110; 5,876,966; 5,876,735; 6,013,268 and 6,080,399, incorporated by reference.

3. Nucleic acid molecules

See U.S. Pat. Nos. 5,593,972; 5,817,637; 5,830,876; 6,063,384; 6,077,663; 5,981,505 and 5,942,235 incorporated by reference.

4. Recombinant Microorganisms which Express Antigens

See U.S. Pat. No. 6,051,237 incorporated by reference.

5. Antibody delivers antigen to antigen presenting cells

See U.S. Pat. No. 5,194,254 incorporated by reference.

10. Therapeutic Antibodies for Treating Infection

1. Shigeoka A O, Pincus S H, Rote N S, Hill H R (1984). Protective efficacy of hybridoma type-specific antibody against experimental infection with group-B Streptococcus. J, Infect. Dis.; 149(3):363-72.

2. Casadevall A, Dcharff M D (1995). Return to the past: the case for antibody-based therapies in infectious diseases. Clin. Infect. Dis.; 21(1):150-61.

11. Desensitization/Tolerization Reagents (Relevant Sections are Incorporated by Reference)

1. Hutchings P, Cooke A (1981). Protection from insulin dependent diabetes mellitus afforded by insulin antigens in incomplete Freind's adjuvant depends on route of administration. J. Autoimmun. 1998; 11(2): 127-30.

2. Harrison (1958). A minireview of prospects for antigen-specific therapy for autoimmune disease; in particular insulin-dependent diabetes, reports various strategies including antigen induced tolerance. Route of administration may produce different results.

3. Javed N H, Gienapp I E, Cox K L, Whitacre C C (1995). Exquisite peptide specificity of oral tolerance in experimental autoimmune encephalomyelitis. J. Immunol.; 155(3): 1599-1605.

4. Oral desensitization or oral tolerance is induced by giving antigenic peptides by the mucosal route, in addition to the oral route. Candidate antigens for multiple sclerosis (MS) and rheumatoid arthritis (RA) are reported by Vischer (1995).

5. Kruisbeek A M, Nieland J D, Jones L A (1992). Mechanism of tolerance induction. Adv. Exp. Med. Biol.; 323: 101-9.

12. Use of Peptides of the Present Invention on Microchips

Microchips that have oligonucleotides or peptides have been developed by many groups or researches for various applications e.g. determining whether genes are present in a biological sample by determining whether DNA molecules in the sample hybridize under conditions wherein hybridization implies a specific degree of homology between a DNA molecule in a sample applied to the microchip and a DNA molecule in the microchip. Microchips are designed so that questions such as “Is the gene for the disease X present in a person?” or “Does the patient have a particular mutation?” or “Is there a specific antigen(s) present in the sample?” can be answered by interpreting the hybridization pattern in the chip, or in the case of antigen or antibody detection, the pattern of antigen-antibody complexing on the microchip. Examples of patents in the microchip area are U.S. Pat. No. 5,861,247 and U.S. Pat. No. 5,770,721. Microchips are sold commercially by Affymetrix, Hyseq and other companies. Licenses are available for microchip technologies through Argonne National Laboratory.

### DOCUMENTS CITED

Ben-Yedidia T et al. (1999) Intranasal administration of peptide vaccine protects human/mouse radiation chimera from influenza infection. Int. Immunol.;11(7):1043-1051.

Casacdevall A, Dcharff M D (1995) Return to the past: the case for antibody-based therapies in infectious diseases. Clin. Infect. Dis.; 21(1): 150-161.

Clin. Exp. Rheum., (1993) 11:121.

Fauchere and Plaska (1983) Eur. J. Med. Chem. 18(4):369-375.

Happ et at. (1981) Proc. Natl. Acad. Sci. USA 78(6);3824-3828.

Harrison L C. (1958) Antigen-specific therapy for autoimmune disease: prospects for the prevention of insulin-dependent diabetes. Mol. Med.; 1(7):722-727.

Hutchings P, Cooke A. (1998) Protection from insulin dependent diabetes mellitus afforded by insulin antigens in incomplete Freund's adjuvant depends on route of administration. J. Autoimmun.;11(9)2:27-130.

Javed N H et al. (1995) Exquisite peptide specificity of oral tolerance in experimental autoimmune encephalomyelitis. J. Immunol; 155(3):1599-1605.

Kruisbeek A M et al. (1992) Mechanism of tolerance induction. Adv. Exp. Med. Biol. 393:101-109.

Lowry O H et al. (1951) Protein measurement with the Folin phenol reagent. J. Biol. Chem. 193:265-275.

Muderspach L et al. (2000) A Phase I trial of a human papillomavirus (HPV) peptide vaccine for women with high-grade cervical and vulvar intraepithelial neoplasia who are HPV 16 positive. Clin. Cancer. Res.; 6(9): 3406-3416.)

O'Brien-Simpson N M et al. (2000) RgpA-Kgp peptide-based immunogens provide protection against Porphyromonas gingivalis challenge in a murine lesion model. Infect. Immun,;68(7):4055-4063.

Parker et al. (1986) Biochemistry 25:5425-5432.

Shigeoka A O et al. (1984) Protective efficacy of hybridoma type-specific antibody against experimental infection with group-B Streptococcus. J. Infect. Dis.; 149(3):363-372.

Trentham et al. (1993) Science 261:1727.

Vischer T L. (1995) Oral-desensitization in the treatment of human immune diseases. Z. Rheumatol. May-June; 54(3):155-157.

U.S. Pat. No. 5,194,254

U.S. Pat. No. 5,593,972

U.S. Pat. No. 5,750,110

U.S. Pat. No. 5,817,637

U.S. Pat. No. 5,830,876

U.S. Pat. No. 5,871,156

U.S. Pat. No. 5,876,735

U.S. Pat. No. 5,876,966

U.S. Pat. No. 5,942,235

U.S. Pat. No. 5,981,505

U.S. Pat. No. 6,013,268

U.S. Pat. No. 6,051,237

U.S. Pat. No. 6,063,384

U.S. Pat. No. 6,077,663

U.S. Pat. No. 6,080,399

U.S. Pat. No. 6,080,409

1. (canceled)

2. (canceled)

3. (canceled)

4. An immunoassay for a targeted molecule to determine if the molecule is present in biological fluid, said immunoassay comprising:
(a) obtaining an immunogenic peptide which produces an immune response in a biologic fluid if the fluid includes a targeted protein, wherein the targeted protein is causative or or associated with a targeted disease or condition, and said peptide comprises the following structure:
i. from 5-10 amino acids in length;
ii. an amino acid sequence which is derived from a protein designated a “targeted” protein.
iii. a net hydrophilic structure as determined by the amino acid sequence of the peptide, said structure located on the surface of the targeted molecule;
iv. an amino acid net sequence homology of 50 percent or less as compared to contiguous amino acid sequences of peptide regions on a comparative protein;
v. an amino acid sequence wherein no more than three contiguous amino acids on the comparative protein are identical; and
vi. an antigenic profile which elicits an immune response specific for the targeted protein.
(b) determining whether the peptide has complexed with an antibody present in the biological fluid from which the presence of the targeted molecule in the fluid is inferred.

5. A diagnostic method for a disease or condition wherein a plurality of an immunogenic peptide which produces an immune response in a biologic fluid if the fluid includes a targeted protein, wherein the targeted protein is causative or, or associated with, a targeted disease or condition, and said peptide comprises the following structure:
i. from 5-10 amino acids in length;
ii. an amino acid sequence which is derived from a protein designated a “targeted” protein.
iii. a net hydrophilic structure as determined by the amino acid sequence of the peptide, said structure located on the surface of the targeted molecule;
iv. an amino acid net sequence homology of 50 percent or less as compared to contiguous amino acid sequences of peptide regions on a comparative protein:
v. an amino acid sequence wherein no more than three contiguous amino acids on the comparative protein are identical; and
vi. an antigenic profile which elicits an immune response specific for the targeted protein
are contacted in a microchip to detect a targeted protein that is causative of, or associated with, the disease or condition, said detection achieved by hybridization of antibodies in the biologic sample to the plurality of peptides on the microchip.

6. A molecule which is specifically reactive with an immunogenic peptide which produces an immune response in a biologic fluid if the fluid includes a targeted protein, wherein the targeted protein is causative or, or associated with, a targeted disease or condition, and said peptide comprises the following structure:
i. from 5-10 amino acids in length;
ii. an amino acid sequence which is derived from a protein designated a “targeted” protein.
iii. a net hydrophilic structure as determined by the amino acid sequence of the peptide, said structure located on the surface of the targeted molecule;
iv. an amino acid net sequence homology of 50 percent or less as compared to contiguous amino acid sequences of peptide regions on a comparative protein;
v. an amino acid sequence wherein no more than three contiguous amino acids on the comparative protein are identical; and
vi. an antigenic profile which elicits an immune response specific for the targeted protein.

7. A molecule which is specifically reactive with a reactive molecule of claim 6.

8. The molecule of claim 6, selected from the group consisting of monoclonal antibodies or immunogenic fragments thereof, recombinant proteins and adhesion proteins.

9. An immunoassay for a targeted protein, said immunoassay comprising:
(a) obtaining a molecule of claim 6; and
(b) determining whether the molecule complexes with the parent protein of the peptide in a biological fluid.

10. A diagnostic method wherein a plurality of targeted proteins are placed in a microchip to detect a microorganism, autoimmune disease, or allergy in a subject from which a biological sample is obtained, said microorganism, autoimmune disease, or allergy is detected by hybridization of targeted proteins to molecules in the biological sample.

11. An immune cell which is specifically reactive with an immunogenic peptide which produces an immune response in a biologic fluid if the fluid includes a targeted protein wherein the targeted protein is causative or, or associated with, a targeted disease or condition, and said peptide comprises the following structure:
i. from 5-10 amino acids in length;
ii. an amino acid sequence which is derived from a protein designated a “targeted” protein.
iii. a net hydrophilic structure as determined by the amino acid sequence of the peptide, said structure located on the surface of the targeted molecule;
iv. an amino acid net sequence homology of 50 percent or less as compared to contiguous amino acid sequences of peptide regions on a comparative protein;
v. an amino acid sequence wherein no more than three contiguous amino acids on the comparative protein are identical; and
vi. an antigenic profile which elicits an immune response specific for the targeted protein.

12. A method for identifying a peptide which functions as a highly specific antigen for a targeted protein, said method comprising:
(a) selecting amino acid sequences of peptides of 5 to 10 amino acids in length by copying a contiguous region of the amino acid sequence of the targeted protein;
(b) synthesizing candidate peptides that have the sequences of step (a);
(c) labeling the peptides at either the NH₂ or COOH and of their amino acid sequence with a detectable label; and
(d) testing by means of immunoassays whether the peptides are specific for the targeted protein.

13. An imaging reagent comprising a molecule of claim 7 and a label.

14. The imaging reagent of claim 13, wherein the label is radioisotopic and, upon binding to microorganisms or diseased tissues highlights the presence of the microorganisms or diseased tissues when scanned with a nuclear medicine scanner.

15. The imaging reagent of claim 13, wherein the label is a paramagnetic label which, upon binding to microorganisms or diseases tissue highlights the presence of the microorganisms or diseased tissue when scanned with a nuclear magnetic resonance (NMR) scanner.

16. The imaging reagent of claim 13, wherein the label comprises a water density which, upon binding to microorganisms or diseased tissues highlights the presence of the microorganisms or diseases tissues when scanned with a CAT scanner.

17. (canceled)

18. (canceled)

19. (canceled)

20. An anti-microbial therapeutic construct comprising a nucleic acid molecule comprising a nucleotide sequence that encodes an immunogenic peptide which produces an immune response in a biologic fluid if the fluid includes a targeted protein, wherein the targeted protein is causative or, or associated with, a targeted disease or condition and said peptide comprises the following structure:
i. from 5-10 amino acids in length;
ii. an amino acid sequence which is derived from a protein designated a “targeted” protein.
iii. a net hydrophilic structure as determined by the amino acid sequence of the peptide, said structure located on the surface of the targeted molecule;
iv. an amino acid net sequence homology of 50 percent or less as compared to contiguous amino acid sequences of peptide regions on a comparative protein;
v. an amino acid sequence wherein no more than three contiguous amino acids on the comparative protein are identical; and
vi. an antigenic profile which elicits an immune response specific for the targeted protein.

21. (canceled)

22. (canceled)


SEQUENCE LISTING (32 sequences)

SEQ ID NO: 1 - protein, 260 aa, Helicobacter pylori
MKTNGHFKDFAWKKCLLGASVGALLVGCSPHIIETNEVALKLNYHPASEKVQALDEKILLLRPAFQYSDNIAKEYENKFKNQTVLKVEQILQNQGYKVINVDSSDKDDFSFAQKKEGYLAVAMNGEIVLRPDPKRTIQKKSEPGLLFSTGLDKMEGVLIPAGFVKVTILEPMSGESLDSFTMDLSELDIQEKFLKTTHSSHSGGLVSTMVKGTDNSNDAIKSALNKIFGSIMQEIDKKLTQKNLESYQKDAKELKGKRNR

SEQ ID NO: 2 - protein, 28 aa, Streptococcus pneumoniae
  MOD_RES (1)..(3): Variable amino acid
XXXIDKXXTQKXLEXXXXDAXELXXXXX

SEQ ID NO: 3 - protein, 28 aa, Helicobacter pylori
MQEIDKKLTQKNLESYQKDAKELKGKRN

SEQ ID NO: 4 - protein, 28 aa, Mycoplasma hominis
  MOD_RES (1)..(2): Variable amino acid
XXEIXKKLXXXXXESXXKDAKELXGXRN

SEQ ID NO: 5 - protein, 12 aa, Helicobacter pylori
MQEIDKKLTQKN

SEQ ID NO: 6 - protein, 10 aa, Helicobacter pylori
KNLESYQKDA

SEQ ID NO: 7 - protein, 12 aa, Helicobacter pylori
QKDAKELKGKRN

SEQ ID NO: 8 - protein, 11 aa, Helicobacter pylori
LPALKENNGKV

SEQ ID NO: 9 - protein, 13 aa, Helicobacter pylori
VTTKGERTFEYNN

SEQ ID NO: 10 - protein, 11 aa, Helicobacter pylori
LEQNEGFKRRV

SEQ ID NO: 11 - protein, 12 aa, Helicobacter pylori
TVDASGKRSISG

SEQ ID NO: 12 - protein, 12 aa, Helicobacter pylori
MQEIDKKLTQKN

SEQ ID NO: 13 - protein, 10 aa, Helicobacter pylori
NTRYDRWAKD

SEQ ID NO: 14 - protein, 15 aa, Helicobacter pylori
INGDKRTGGKPNTPE

SEQ ID NO: 15 - protein, 10 aa, Helicobacter pylori
SSSEYEKLKA

SEQ ID NO: 16 - protein, 12 aa, Helicobacter pylori
KEQIIEAKGPDV

SEQ ID NO: 17 - protein, 10 aa, Helicobacter pylori
QQTHRKINRP

SEQ ID NO: 18 - protein, 10 aa, Helicobacter pylori
KQAEEANKTP

SEQ ID NO: 19 - protein, 10 aa, Helicobacter pylori
KTPDKPDKVW

SEQ ID NO: 20 - protein, 9 aa, Homo sapiens
QGQDVRQPG

SEQ ID NO: 21 - protein, 5 aa, Homo sapiens
GRDGE

SEQ ID NO: 22 - protein, 10 aa, Homo sapiens
GGFDEKAGGA

SEQ ID NO: 23 - protein, 7 aa, Homo sapiens
WPGLPGA

SEQ ID NO: 24 - protein, 28 aa, Homo sapiens
GLPGTPGTDGPKGASGPAGPPGAQGPPG

SEQ ID NO: 25 - protein, 15 aa, Homo sapiens
GPEGAPGKDGGRGLT

SEQ ID NO: 26 - protein, 18 aa, Homo sapiens
GEVGPPGPAGSAGARGAP

SEQ ID NO: 27 - protein, 22 aa, Homo sapiens
TGPKGARGAQGPPGATGFPGAA

SEQ ID NO: 28 - protein, 13 aa, Homo sapiens
NGNPGPPGPPGPS

SEQ ID NO: 29 - protein, 8 aa, Homo sapiens
LQGPAGPP

SEQ ID NO: 30 - protein, 25 aa, Homo sapiens
AVGAPGAPGPPGSPGPAGPTGKQGD

SEQ ID NO: 31 - protein, 10 aa, Homo sapiens
KGHRGFTGLQ

SEQ ID NO: 32 - protein, 21 aa, Homo sapiens
IGPPGPRGRSGETGPAGPPGN